(19) United States
(12) Patent Application Publication
(10) Pub. No.: US 2015/0118361 A1
(43) Pub. Date: Apr. 30, 2015

(54) Use of Ferulic Acid Esterase to Improve Performance in Monogastric Animals

(71) Applicant: Kemin Industries, Inc., Des Moines, IA (US)

(72) Inventors: Sathishkumar Jayaraman, Chennai (IN); Rajalekshmi Mukkalil, Kerala (IN); Haridasan Chirakkal, Chennai (IN)

(21) Appl. No.: 14/522,968

(22) Filed: Oct. 24, 2014

(30) Foreign Application Priority Data
Oct. 25, 2013 (IN) 3173DEL2013

(51) Int. Cl. A23K 1/165; A23K 1/18; C12N 9/24; C12N 9/26; C12N 9/18; C12N 9/42

(52) U.S. Cl. CPC A23K 1/1656; C12N 9/18; C12N 9/2437; C12N 9/248; C12N 9/2411; A23K 1/1833; A23K 1/184; C12Y 301/01073
USPC 426/61

(57) ABSTRACT

The presence of non-starch polysaccharides (NSP) in the plant cell wall reduces the digestibility and limits the apparent metabolizable energy (AME) and performance of animals. The main chain degrading enzymes, especially xylanase, cellulase and glucanase play an important role in improving the digestibility of NSP in the feed. Ferulic acid esterase (FAE) breaks the ferulate cross linkages in the plant cell wall, and aids the main chain hydrolases to further degrade the plant cell wall. The present study investigated the synergy of FAE in combination with main chain degrading enzymes in improving the AME of birds fed with high fiber diet. The addition of FAE improves the access of main chain degrading enzymes, digestibility of high fiber diet, AME in layers and broilers, Body weight and reduces FCR in broilers.

### BACKGROUND OF THE INVENTION

The present invention relates generally to feeding of monogastric animals and, more specifically, to the use of ferulic acid esterase to improve apparent metabolizable energy and performance.

The use of exogenous enzymes in animal nutrition plays a significant role in improving the nutrient utilization and growth performance of poultry. When birds are fed with cereal based diets, the presence of non-starch polysaccharides (NSP) present in cell walls increases the in vivo viscosity of the digesta. This results in the reduction in the apparent metabolizable energy (AME) (see, for example, Annison, G. Relationship between the levels of soluble non-starch polysaccharides and the apparent metabolizable energy of wheats assayed in broiler chickens J. Agric. Food Chem., 1991, 39 (7), pp 1252-1256).

The use of xylanase, cellulase and glucanase in degradation of NSP is well documented. However the cell wall biodegradability is also affected by the presence of ferulic acid (FA) which cross-links the cell wall polymers. Ferulic acid is the major phenolic acid found esterified to carbohydrates in the plant cell wall. The presence of ferulic acid esters in plant material can reach up to 2.5% (w/w) of cell walls (Mastihuba, V., L. Kremnicky, M. Mastihubov, J. L. Willett, and G. L. Cote. 2002. A spectrophotometric assay for feruloyl esterases. Analytical Biochemistry 309: 96-101).

Ferulic acid esterase (FAE) is an enzyme which has the ability to hydrolyze the ester bond between the xylan polysaccharide and the ferulate or diferulates present in the plant cell walls (Christov, L. P., Prior, B. A., 1993. Esterases of xylan-degrading micro-organisms: production, properties and significance. Enzyme Microbiol. Technol. 15: 460-475). The synergy of FAE with xylanase is well documented (Faulds, C. B, Williamson, G. (1995). Release of ferulic acid from wheat bran by a ferulic acid esterase (FAE-III) from Aspergillus niger. Appl Microbiol Biotechnol. 43:1082-1087). It is also believed that, by breaking the ferulate linkages in the plant cell wall, FAE aids the main chain hydrolases to further degrade the plant cell wall. This result in increase in sugars such as D-glucose, D-xylose and L-arabinose released after partial or complete hydrolysis of NSP.

### SUMMARY OF THE INVENTION

The present invention consists of a method of improving the apparent metabolizable energy and performance from a diet in an animal by adding an efficacious amount of a ferulic acid esterase to the diet and/or supplemented along with main chain degrading enzymes. The use of a ferulic acid esterase is shown in a preferred embodiment to improve the AME of broilers fed a high fiber diet.

The present invention also consists of a method of reducing the amount of main chain degrading enzymes needed to improve the AME of diets by adding an efficacious amount of a ferulic acid esterase to the diet. The addition of a ferulic acid esterase can reduce the amount of main chain degrading enzymes by between 20% and 80%.

### BRIEF DESCRIPTION OF THE FIGURES

FIG. 1 is a graphical representation of the effect of enzyme supplementation on AME of broiler birds fed on high fiber diet. Groups that are significantly different from each other at P<0.05 are indicated by different indices (n=12). The values are expressed ad mean±S.E.

FIG. 2 is a graphical representation of the effect of enzyme supplementation on AME of broiler birds fed on high fiber diet. Groups that are significantly different from each other at P<0.05 are indicated by different indices (n=12). The values are expressed ad mean±S.E

FIG. 3 is a graphical representation on the effect of supplementation of FAE along with main chain degrading enzymes on in vitro sugar release from a natural feed substrate. Groups that are significantly different from each other at P<0.05 are indicated by different indices (n=3). The values are expressed ad mean±S.E

FIG. 4 is a graphical representation of effect of supplementation of FAE on AME of broiler birds; groups that are significantly different from each other at P<0.05 are indicated by different indices (n=12). The values are expressed ad mean±S.E

FIG. 5 is a graphical representation of the effect of supplementation of Prototypes 3 and 4 on AME of layer cockerels; groups that are significantly different from each other at P<0.05 are indicated by different indices (n=12). The values are expressed ad mean±S.E

FIG. 6 is a graphical representation of the effect of supplementation of Kemzyme XPF on AME of layer cockerels; groups that are significantly different from each other at P<0.05 are indicated by different indices (n=12). The values are expressed ad mean±S.E

FIG. 7 is a graphical representation of the effect of supplementation of Kemzyme XPF on body weight of broilers at the age of 42 days; groups that are significantly different from each other at P<0.05 are indicated by different indices (n=9); the values are expressed as mean±SE.

FIG. 8 is a graphical representation of the effect of supplementation of Kemzyme XPF on FCR of broilers at the age of 42 days; groups that are significantly different from each other at P<0.05 are indicated by different indices (n=9); the values are expressed as mean±SE.

### DESCRIPTION OF THE INVENTION

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

Unless otherwise defined, all technical and scientific terms used herein have the same meaning as commonly understood by one of ordinary skill in the art to which this invention belongs.

All publications, U.S. patent applications, U.S. patents and other references cited herein are incorporated by reference in their entireties.

As used herein, the term “meat-type poultry” refers to any avian species that is produced or used for meat consumption as understood by one skilled in the art. Examples of such avian species include, but are not limited to, chickens, turkeys, ducks, geese, quail, pheasant, ratites, and the like.

As used herein, the term “apparent metabolizable energy” refers to the gross energy of the feed consumed minus the gross energy contained in the feces, urine and the gaseous products of digestion. For poultry, the gaseous products are usually negligible.

As used herein, the term “ferulic acid esterase” refers to an enzyme that catalyzes the chemical reaction cleaving a feruloyl polysaccharide into a ferulate and a polysaccharide. The enzyme belongs to the family of hydrolases, specifically those acting on carboxylic ester bonds. The systematic name of this enzyme is feruloyl esterase, Other names in common use include hydroxycinnamoyl esterase, hemicullulase accessory enzyme, and cinnamoyl ester hydrolase.

As used herein, the term “main degrading enzyme” refers to the main enzyme included in the feed for an animal that catalyzes the degradation of main components of the feed. Main degrading enzymes include cellulases, xylanases, glucanases and amylases.

In preferred embodiments of the present invention, the dosage of ferulic acid esterase ranges from 20 U/kg to 200 U/kg of feed and all values between such limits, including 30, 40, 50, 60, 70, 80, 90, 100, 110, 120, 130, 140, 150, 160, 170, 180 and 190 U/kg.

In preferred embodiments of the present invention, reducing the main chain degrading enzymes necessary to extract a given amount of the apparent metabolizable energy from a diet in an animal, comprises the step of adding an efficacious amount of a ferulic acid esterase to the diet such that the reduction is by between 20% and 80% and all values between such limits, including 25, 30, 35, 40, 45, 50, 55, 60, 65, 70, and 75%.

In preferred embodiments of the present invention, enhancing the apparent metabolizable energy of the diet, comprises the step of adding an efficacious amount of a ferulic acid esterase to the diet such that the enhancement is by between 0.1% and 100% and all values between such limits, including 0.2, 0.3, 0.4, 0.5, 0.6, 0.7, 0.8, 0.9, 1, 2, 3, 4, 5, 6, 7, 10, 11, 12, 13, 14, 15, 16, 17, 18, 19, 20, 25, 30, 35, 40, 45, 50, 55, 60, 65, 70, 75, 80, 85, 90 and 95%.

In preferred embodiments of the present invention, improving the digestibility of high fiber diets, comprises the step of adding an efficacious amount of a ferulic acid esterase to the diet such that the improvement is by between 0.1% and 100% and all values between such limits, including 0.2, 0.3, 0.4, 0.5, 0.6, 0.7, 0.8, 0.9, 1, 2, 3, 4, 5, 6, 7, 10, 11, 12, 13, 14, 15, 16, 17, 18, 19, 20, 25, 30, 35, 40, 45, 50, 55, 60, 65, 70, 75, 80, 85, 90 and 95%.

In preferred embodiments of the present invention, improving the performance of animal fed a diet, comprises the step of adding an efficacious amount of a ferulic acid esterase to the diet such that the improvement is by between 0.1% and 100% and all values between such limits, including 0.2, 0.3, 0.4, 0.5, 0.6, 0.7, 0.8, 0.9, 1, 2, 3, 4, 5, 6, 7, 10, 11, 12, 13, 14, 15, 16, 17, 18, 19, 20, 25, 30, 35, 40, 45, 50, 55, 60, 65, 70, 75, 80, 85, 90 and 95%.

As used herein, the term “layer” refers to a member of the avian species that is used primarily for the production of eggs.

As used herein, the term “broiler” refers to any immature chicken produced or eventually used for meat consumption.

As used herein, the term “poultry diet” refers to a diet that can be administered to a member of the avian species to promote and maintain growth of the bird. A poultry diet can contain sources of protein, vitamins, minerals, energy such as fat, carbohydrates, and additional protein, antibiotics, and other substances or compounds known to be included in animal feeds, in particular, poultry feeds. Poultry diet is inclusive of, but not limited to, a starter diet, a grower-type diet, and a finisher-type diet. A “starter diet” refers to a diet that can be administered to an animal starting from birth or hatch until a desired age and/or weight is obtained. A “grower-type diet” refers to a diet that can be administered to an animal upon completion of the starter growth phase. A “finisher-type diet” refers to a diet that can be administered to an animal during the period of development through the time of slaughter.

As used herein, the terms “growth” or “growth performance” refer to increases in either, or both, weight and size (e.g., height, width, diameter, circumference, etc.) over that which would otherwise occur without implementation of the methods and/or administration of the compositions of the present invention. Growth can refer to an increase in the mass (e.g., weight or size) of the entire animal or of a particular tissue (e.g., muscle tissue in general or a specific muscle). Alternatively, growth can indicate a relative increase in the mass of one tissue in relation to another, in particular, an increase in muscle tissue relative to other tissues (e.g., adipose tissue). Growth further relates to nutritional status and disease resistance wherein improvement of nutritional status and/or increase in disease resistance is also indicative of improved growth performance.

As used herein, the term “feed conversion ratio” refers to a measure of an animal's efficiency in converting feed mass into increases for the desired output. For animals raised for meat, such as swine and broilers, the output is the mass gained by the animal. For animal raised for eggs, such as layers, the ouput is the mass of the eggs produced by the layer. Specifically, feed conversion ratio is the mass of food eaten divided by the output, all over a certain period. Other terms in common use are feed conversion rate and feed conversion efficiency.

In view of the foregoing, embodiments according to the present invention relate to methods of growing monogastric animals, particularly poultry and swine, comprising feeding an animal feed diet wherein the feed further comprises ferulic acid esterase and is added to the diet in an amount effective to enhance the apparent metabolizable energy of the diet, and/or reduce the dosage of main degrading enzyme(s) of the diet, and/or improve the digestibility of high fiber diets, and/or improve the performance of animal fed the diet. The poultry diet can be an animal feed which includes sources of protein, for example, soybean meal, fish meal, blood meal, poultry by-product (ground poultry offal), meat meal, wheat-meal, rapeseed, canola and combinations of the same. The animal feed further includes carbohydrates, for example, corn, oats, barley, sorghum, or combinations of the same that can be ground into a meal for use in the animal feed. Additionally, the animal feed can include vitamins, minerals, fat, antibiotics, and other substances or compounds as necessary or desired. Non-limiting examples of animal feed poultry diets include cereal-based feeds including cereals such as barley, corn, soya, wheat, triticale, and rye. Corn-soybean, wheat-soybean, and wheat-corn-soybean, sorghum-soybean, and corn-sorghum-soybean represent other non-limiting examples of suitable animal feeds according to the present invention.

Ferulic acid esterase for practicing the present invention can be obtained by growing a host cell which contains nucleic acid sequences encoding a ferulic aid esterase, under conditions which permit expression of the encoded ferulic aid esterase, optionally filtering the medium to remove the cells and collecting and concentrating the remaining supernatant by ultrafiltration to obtain the ferulic aid esterase. Beneficiary co-factor(s) can also be obtained.

As provided herein, a ferulic aid esterase enzyme may be produced by culturing a host cell as described above under conditions that permit expression of the encoded ferulic aid esterase, and collecting the expressed ferulic aid esterase. The host cell may be cultured under conditions in which the cell grows, and then cultured under conditions which cause the expression of the encoded ferulic aid esterase, or the cells may be caused to grow and express the encoded ferulic aid esterase at the same time. Such conditions are well known to one of skill in the art and may vary with the host cell and the amount of enzyme expression level desired.

The ferulic aid esterase should be present in an amount at least sufficient to achieve the intended effect, but the upper limit to the amount of ferulic aid esterase can be determined based upon achieving the intended effect. In some embodiments, the animal feed comprises from about 0.01% to about 20% ferulic aid esterase by weight. Additionally, ferulic aid esterase used in practicing the present invention can be in crude form or in pure form. ferulic aid esterase in crude form can be prepared, for example, by separating bacterial cells which produce the ferulic aid esterase from their liquid growth media, the liquid growth media comprising crude ferulic aid esterase. Alternatively, the cells can be lysed (chemically or physically) in a liquid growth media to produce a crude, cell free extract. Other means of preparing such an extract will be apparent to persons skilled in the art. The crude ferulic aid esterase can be included in the feed in any form compatible therewith, such as in an aqueous form or in lyophilized form. In some embodiments, the crude ferulic aid esterase is in the lyophilized form.

Pure (or substantially pure) ferulic aid esterase can be obtained by separating the crude ferulic aid esterase described above into its individual constituents, in accordance with known techniques. Numerous suitable separation procedures, such as column chromatography, are known to persons skilled in the art. The individual constituent proteins can be screened for their ability to degrade ferulic aid-containing material, and that constituent which best degrades ferulic aid esterase-containing material comprises the ferulic aid esterase. Like the crude ferulic aid esterase, the pure ferulic aid esterase can be employed in any suitable form, including aqueous form and lyophilized form.

Embodiments of the present invention further relate to methods of improving the efficiency of feed utilization of an animal feed comprising feeding an animal feed wherein the feed further comprises ferulic aid esterase in an amount effective to improve the efficiency of feed utilization of an animal feed provided to meat-type poultry. The animal feed can include the animal feeds as described above and, in particular embodiments can be corn-soybean meal.

The animal feed of the present invention comprises ferulic aid esterase in an amount at least sufficient to achieve the intended effect, wherein the upper limit to the amount of ferulic aid esterase can be determined based upon achieving the intended effect. The animal feed supplement added to the animal feed can comprise up to 100% ferulic aid esterase by weight. The animal feed comprising the supplement comprises from about 5% to about 25% ferulic aid esterase by weight.

Any animal is a suitable subject for the present invention, however, the present invention is preferably employed with monogastric animals. Suitable subjects can be of any age range including neonatal animals, developing animals, and mature animals. In some embodiments, the suitable subject can be an avian, preferably a chicken. In other embodiments the suitable subject can be a chicken. In still other embodiments, the suitable subject can be an immature, developing, or mature bird. In other embodiments, the suitable subject can be a chicken that is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 days old, or within any range of these numbers. Thus, the present invention provides a variety of different feeds, including pet feed, poultry feed, and pig feed.

The animal feed supplement of the present invention can also enable a conventional animal feed to be modified by reducing its energy, and/or protein, and/or amino acid content while simultaneously maintaining the same nutritional levels of energy, protein, and amino acids available to the animal. Consequently, the amounts of costly energy and protein supplements typically included in an animal feed can be reduced as compared to conventional feeds.

### Example 1

### FAE Enhances Apparent Metabolizable Energy in a Diet

### Materials and Methods

Enzymes and Chemicals.

Ferulic acid esterase [12.5 U/mg on substrate-(O-{5-O-[(E)-feruloyl]-a-L-arabinofuranosyl}-(133)-O-b-D-xylopyranosyl-(134)-D-xylopyranose), FAXX] Other enzymes used in the AME trial include cellulase, xylanase, glucanase and amylase obtained from Kemin Industries South Asia Private limited manufacturing facility, Gummidipoondi

HPLC Conditions and Equipment.

The HPLC column used was a C18 Column (Phenomenex) 250 mm×4.6 mm with 5 μm particle size. The mobile phase is methanol: water: acetic acid (35:65:1) at a flow rate of 1.0 ml/min in isocratic elution mode. The oven temperature was set at 40° C. A sample volume of 20 μl was used at a run time of 20 min. The detector was set to 320 nm.

Ferulic Acid Esterase Assay.

Ferulic acid esterase activity was assayed using MFA as a substrate as described by Shin, et al. (Shin, H. D., R. R Chen. (2006). Production and characterization of a type B feruloyl esterase from Fusarium proliferatum NRRL 26517. Enzyme and Microbial Technology 38: 478-485) with a few modifications. The assay was carried out in 0.5 mL 50 mM sodium phosphate buffer (pH 6.5) containing 1 mM methyl ferulate at 40° C. for 30 min, and then the liberated free ferulic acid was analyzed by HPLC as described above wherein the final reaction volume was made up to 1 mL with the buffer. The substrate control used in the assay contained only methyl ferulate without FAE. One unit of FAE activity was defined as the amount of enzyme that liberated 1 μmol ferulic acid/min in the above assay conditions.

Statistical Analysis.

All analyses were carried out in triplicate, and the mean was calculated. Data are expressed as mean±SD. Statistical analysis was performed using ANOVA with STATGRAPHICS Plus. Differences at P<0.05 were considered significant.

Preparation of Prototype I Enzymes.

Prototype I was prepared using the combination of main chain degrading enzymes amylase, xylanase, cellulase and glucanase.

Activity Profile of Prototype I Enzymes.

The Prototype I enzymes included cellulase, xylanase, glucanase and amylase. The activity profile of individual enzymes on specific substrates is shown in Table 1. All assays were performed following standard protocols. The FAE activity was determined using methyl ferulate as substrate as described above.

Along with the Prototype I enzymes, FAE was added at the dosage of 20 U and 40 Upper kg of feed.

TABLE 1
Activity profile of Prototype I enzymes

| Enzymes | Enzyme activity/g of Prototype I* |
|---|---|
| Cellulase | 6351.84 ± 48.07 |
| Xylanase | 42358.98 ± 192.3 |
| Glucanase | 8426.18 ± 119.04 |
| Amylase | 1394 ± 3 |

*The enzyme assays were performed on specific substrates, following standard techniques

Synergy Between Prototype I and FAE.

The synergy between Prototype I enzymes and FAE in release of ferulic acid from destarched wheat bran (DSWB) was tested substantially as described in Fualds, et al. (Faulds, C. B, Williamson, G. (1995). Release of ferulic acid from wheat bran by a ferulic acid esterase (FAE-III) from Aspergillus niger. Appl Microbiol Biotechnol. 43:1082-1087). The Prototype I (100 mg) was dissolved in 100 mL of sodium phosphate buffer (pH 6.5) and 100 uL of the enzyme solution was used for the assay. The groups included FAE (20 uL, 25 U/mg on FAXX), Prototype I enzymes (100 uL) and combination of FAE and Prototype I enzymes. The above mentioned quantity of enzymes was added to 25 mg DSWB and final volume of reaction mixture was made up to 1 mL using sodium phosphate buffer (pH −6.5). The reaction was incubated at 40° C. for 1 h. Then the tubes were kept in boiling water bath for 10 min to stop the reaction. The tubes were centrifuged at 10000 rpm for 10 min and the released ferulic acid was read using HPLC at 320 nm. The tube with DSWB, containing only sodium phosphate buffer without any enzymes was used as control.

Birds and Trial Design.

The AME trials were carried out at Kemin R&D farm, Gummidipoondi. Cobb 400 (35 days old) broiler birds were housed in metabolic cages and fed a commercial broiler finisher diet prior to the commencement of the trial.

Nutrient Composition of Feed.

The diet was reformulated to have increased fiber content of 4.2% as against 3.5% fiber in normal broiler finisher feed with reduction in energy of 80 Kcal/kg. The feed composition is shown in Table 2 and the nutrient composition is shown in Table 3.

TABLE 2
Feed composition of the High fiber diet used in the AME trials

| Ingredients | Concentration (Kg/ton) |
|---|---|
| Maize | 604.22 |
| Soya Meal | 278.57 |
| Sunflower meal^{$} | 43.13 |
| Calcite/LSP | 16.65 |
| DCP | 8.95 |
| Rice Bran oil | 35.53 |
| DL-Methionine | 2.46 |
| L-Lysine | 2.39 |
| Kemzyme PG 5000 | 0.1 |
| Soda bicarbonate | 1 |
| Salt | 3 |
| Additives(g) | |
| Vitamin premix Provit plus | 0.5 |
| Choline Chloride | 1 |
| Superliv | 0.5 |
| BMD | 0.5 |

^{$}Sunflower meal was used in the formulation to increase the fiber content of the feed.

TABLE 3
Nutrient composition of diet used in the AME trials

| Nutrient | |
|---|---|
| composition | value |
| ME | 3070 Kcal/kg |
| CP | 19% |
| CF | 4.20% |
| EE | 6.08% |
| Ca | 0.92% |
| Av P | 0.42% |
| Lysine | 1.20% |
| Methionine | 0.55% |

Treatment Groups—Trial I.

The treatment groups included in the trial I was shown in Table 4.

TABLE 4
Treatment groups - Trial I

| Treatment | | | Dosage | |
|---|---|---|---|---|
| groups | Diet | Enzyme | FAE^{$} | Prototype I* |
| Control | High fiber | No enzyme | — | — |
| | diet | | | |
| Treatment 1 | High fiber | Prototype I | — | 500 g/ton of feed |
| | diet | | | |
| Treatment 2 | High fiber | Ferulic acid | 20 U/kg | 500 g/ton of feed |
| | diet | Esterase + | | |
| | | Prototype I | | |
| Treatment 3 | High fiber | Ferulic acid | 40 U/kg | 500 g/ton of feed |
| | diet | Esterase + | | |
| | | Prototype I | | |

*The Prototype I contains cellulase, amylase and glucanase and Xylanase as given in Table 1
^{$}FAE activity was determined using methyl ferulate as substrate

Treatment Groups—Trial II.

In trial II, Prototype I was used at a lower dosage (Table 5). Rest of the experiment design was similar to that of Trial I.

TABLE 5
Treatment groups - Trial II

| Treatment | | | Dosage | |
|---|---|---|---|---|
| groups | Diet | Enzyme | FAE^{$} | Prototype 1* |
| Control | High fiber | No enzyme | — | — |
| | diet | | | |
| Treatment 1 | High fiber | Prototype I | — | 250 g/ton of feed |
| | diet | | | |
| Treatment 2 | High fiber | Ferulic acid | 20 U/kg | 250 g/ton of feed |
| | diet | Esterase + | | |
| | | Prototype I | | |
| Treatment 3 | High fiber | Ferulic acid | 40 U/kg | 250 g/ton of feed |
| | diet | Esterase + | | |
| | | Prototype I | | |

*The Prototype I contains cellulase, amylase and glucanase and Xylanase as given in Table 1
^{$}FAE activity was determined using methyl ferulate as substrate

AME Trial.

AME trials were done in broiler birds of 5 weeks age. Each trial was done in 48 birds in 4 treatment groups, containing 12 birds per replicate in metabolic cages. The birds were kept in individual cages over an excreta collection tray. The adaptation in the cage was done for 3 days with ad libitum diets and water. Before feeding the reformulated diets, birds were starved for 24 hours to empty their gut contents. After 24 h, the birds were fed with 50 g of feed according to the treatment groups as mentioned in Table 4 and 5. The excreta were collected from each bird for exactly 36 h after feeding and the excreta samples are dried at 50° C. for 48 h. The dry weight of excreta was measured and the samples were used for gross energy measurements using Parr 6300 Bomb calorimeter (Parr Instrument Company, USA). The gross energy of the feed samples was measured and AME (Kcal/g) was calculated as shown below.

$$AME = \frac{\begin{matrix}Total Gross Energy Intake ( Kcal ) - \\ Total Gross Energy excreted ( Kcal )\end{matrix}}{Total Feed Intake ( g )}$$

### Results

The synergy between FAE and Prototype I enzymes is shown in Table 7. The use of Prototype I enzymes alone released negligible amount of ferulic acid from substrate. However, FAE in combination with Prototype I enzymes significantly improved ferulic acid release, as compared to either FAE or Prototype I (Table 7). This demonstrates the synergy of FAE with xylanase and other main chain degrading enzymes.

TABLE 7
Synergy between FAE and Prototype I enzymes in the release of ferulic
acid from DSWB

| | Ferulic acid released |
|---|---|
| Groups | from DSWB (uM) |
| FAE | 35 ± 2 |
| Prototype I | 3.73 ± 0.40 |
| Prototype I + | 441.54 ± 3.10 |
| FAE | |
| Control | 2.44 ± 0.09 |

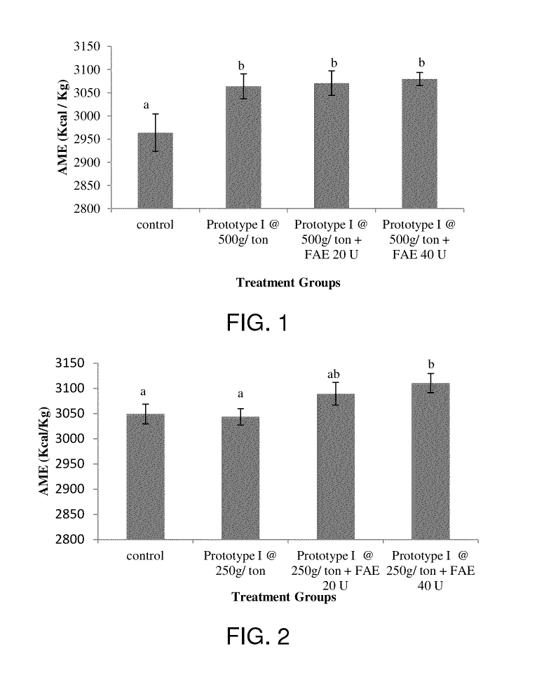

The AME data of the trial I using Prototype enzymes at 500 g/ton of feed with FAE at two different dosages is shown in FIG. 1, where groups that are significantly different from each other at P<0.05 are indicated by different indices (n=10). The groups supplemented with Prototype enzymes improved AME significantly as compared to control. No statistical difference was observed between the group supplemented with Prototype enzyme and group supplemented with Prototype enzyme with FAE at 20 U/kg and 40 U/kg respectively. Only numerical difference was observed between the groups added with Prototype enzymes and FAE.

The AME of the trial II using Prototype enzymes at 250 g/ton of feed with FAE at two different dosages is shown in FIG. 2, where groups that are significantly different from each other at P<0.05 are indicated by different indices (n=10). There was no significant difference in AME observed between the control group and the group supplemented with Prototype I. However, the addition of FAE to the Prototype I improved the AME of broiler birds. The supplementation of FAE (20 U) to the Prototype I improved 40 Kcal/kg of AME and FAE (40 U) improved (P<0.05) 61 Kcal/kg AME in broiler birds. Though the same diet formulation was used in both AME trials, the AME values of control groups was observed to be different in two trials, This might be attributed to the quality of feed grains used in the trial.

Dietary fiber includes polysaccharides that are not digested by the endogenous enzymes of the digestive tract. These polysaccharides include resistant starch and non-starch polysaccharides (NSP). NSP are generally classified as water soluble (water soluble pectins, glucans and arabinoxylans) and water insoluble (lignin, cellulose, hemicellulose and pectic substances). Lignin is cross-linked to arabinoxylans by ferulate molecules. These ferulates are esterified to arabinose units of arabinoxylans, and some ferulate esters combine to form diferulates to cross-link arabinoxylan chains, with a portion of these diferulates also becoming linked to lignin (Jung, H. G. D. R. Mertens and R. L. Phillips (2011). Effect of reduced ferulate-mediated lignin/arabinoxylan cross-linking in corn silage on feed intake, digestibility, and milk production. J. Dairy Sci. 94:5124-5137). It has been proposed that crosslinking of lignin and arabinoxylans will impede cell wall digestibility by placing lignin in very close proximity to the polysaccharides and preventing physical access by hydrolytic microbial enzymes.

The use of ferulic acid esterase might be useful in breaking these cross links and provides access for other hydrolytic enzymes to act on the polysaccharides, thereby improving the nutritional quality of the animal feed. FAE used in this trial was a recombinant enzyme from Clostridium thermocellum, which is >95% purity. Pure FAE was used in this trial to determine the effective dosage of FAE in improving the AME of the broiler birds fed with high fiber diet.

Sunflower meal is used in this trial to increase the fiber content of the feed as it contains high NSP content. It was reported that the soluble and insoluble constituents of NSP content are 4.5 and 23.1%, respectively (Senkoylu N. and N. Dale (2006). Nutritional Evaluation of a High-Oil Sunflower Meal in Broiler Starter Diets. J. Appl. Poult. Res. 15:40-47).

The trial I with Prototype I at 500 g/ton of feed and FAE did not improve the AME compared to groups supplemented with Prototype enzymes (FIG. 1). This might be due to the fact, the Prototype I at 500 g/ton itself compensated the reduced energy of 80 Kcal in the diet and the contribution of additional energy from FAE was minimal. This prompted us to use the Prototype I enzymes at reduced dosage to observe the synergy between FAE and Prototype I enzymes. In trial II, Prototype I was used at 250 g/ton of feed (Table 5) to establish the synergy between FAE and main chain degrading enzymes in improving the AME of the broiler birds. It is evident from the graph (FIG. 2) that FAE provides improved access to the main chain degrading enzymes and improves the available energy from feed, when used at half the dosage. This also demonstrates the role of FAE as a key enzyme in improving the nutritional quality of feed. This also provides the information, that the usage of main chain degrading enzymes could be reduced, when used in combination with FAE, to provide improved economic benefits.

### Example 2

### FAE Releases Sugar from a Starch Substrate

### Materials and Methods

Preparation of Destarched Wheat Bran (DSWB).

Food grade wheat bran was obtained from M/s Bannari Amman Flour Mill (Chennai, India). The wheat bran was destarched by incubation in 0.25% (w/v) potassium acetate for 10 minutes at 95° C. The treated bran was then washed thoroughly with water. After each wash, the water sample was tested for starch using iodine solution. The washing continued till the water sample tested negative for starch. The DSWB sample was then dried in a hot air oven at 50° C. overnight to remove all traces of moisture. The dried sample was ground using a blender and then passed through sieves of sizes 0.25 to 0.5 mm. The DSWB particles with a particle size of 0.25-0.5 mm were used as the substrate for enzyme assays.

Enzymatic Digestion.

A quantity of 1 g of DSWB was weighed and transferred into clean, grease free conical flask (250 ml). Phosphate buffer (20 mL, 50 mM, 6.5 pH) was initially added to it. The quantity of enzymes was measured and added according to the respective experiment groups. The enzyme activities of base formulation was shown in Table 8. The reaction mixture was then incubated at a pH of 6.5 at a temperature of 40° C. for 3 hour. After this period, the reaction was arrested by heating at 100° C. for 5 minutes. This was then centrifuged at 10,000 rpm for 10 minutes to remove DSWB particles. The supernatant was read for reducing sugar using Nelson Somogyi method.

TABLE 8
Enzyme activity of base formulation

| | Base |
|---|---|
| | formulation |
| Enzymes | (BF) (u/g) |
| Cellulase | 14,138 ± 20 |
| α-amylase | 680 ± 8 |
| β-glucanase | 3,413 ± 49 |
| Xylanase | 44,069 ± 1068 |

*Values are expressed as mean ± SD, n = 3

Enzyme Assay.

All the enzyme assays were performed using standard methods. FAE—Ferulic acid esterase, Depol 740 L obtained from Biocatalyst Ltd, UK. FAE activity was determined using methyl ferulate as the substrate by an HPLC method.

### Results

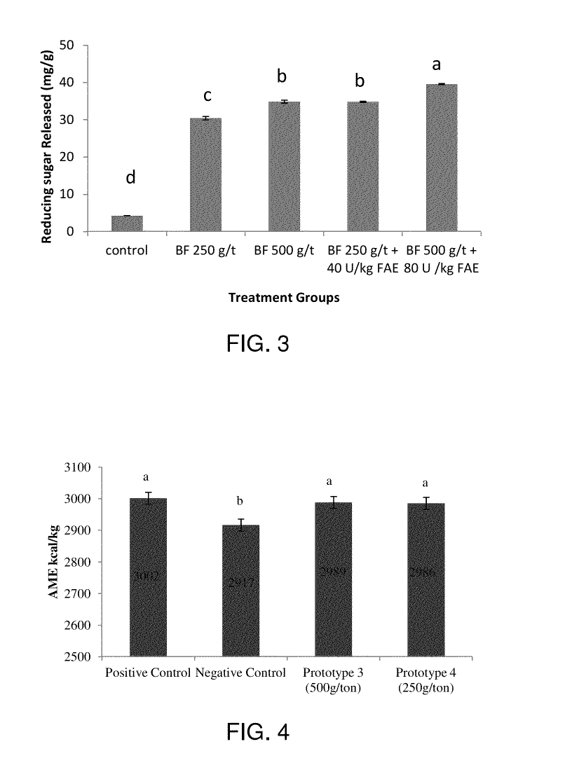

The reducing sugar released from the enzyme supplemented groups is significantly higher than the control group (FIG. 3). There was clear dose response observed between the base formulation at 250 and 500 g/ton. The addition of FAE 40 U/kg to BF-250 g/t improved sugar releases significantly and was equivalent to BF supplemented at 500 g/ton. The supplementation of BF −500 g/t along with 80 U/kg FAE had significantly higher sugar released compared to all other treatment groups. From this data, it is evident that, FAE not only improves the access of main chain enzymes like cellulase, glucanase, and xylanase, but also reduces the dosage of main chain enzymes.

### Animal Trials

Enzyme Assay.

All the enzyme assays were performed using standard methods. FAE activity was determined using methyl ferulate as the substrate by an HPLC method.

Prototypes.

Prototypes 3 and 4 were formulated with BAN 800 and slow release amylase in equal ratio along with FAE and other NSPases (Tables 9a and 9b). Prototype 4 was different from prototype 3 with the double concentration of FAE. The respective enzyme activities are shown in Table 2a and 2b.

TABLE 9a
Enzyme activities (analyzed) of the prototypes

*Enzyme Activity (U/g)

| Enzymes | Prototype 3 | Prototype 4 |
|---|---|---|
| Cellulase | 13,570 ± 211 | 14,138 ± 20 |
| α-amylase | 690 ± 7 | 680 ± 8 |
| β-glucanase | 3,317 ± 99 | 3,413 ± 49 |
| Xylanase | 39,932 ± 2447 | 44,069 ± 1068 |
| FAE | 85 ± 9 | 166 ± 17 |

*Values are expressed as mean ± SD, n = 3

TABLE 9b
Enzyme activities of the prototypes in treated feed

*Enzyme Activity (U/g)

| Enzymes | ^{#}Prototype 3 | ^{#}Prototype 4 |
|---|---|---|
| Cellulase | 6785 | 3534.5 |
| α-amylase | 345 | 170 |
| β-glucanase | 1658.5 | 853 |
| Xylanase | 19966 | 11017 |
| FAE | 42.5 | 41.5 |

*Calculated based on the analyzed values shown in Table 9b
^{#}Prototypes 3 and 4 were administered at the dosage of 500 g/ton and 250 g/ton respectively

Metabolic Trial.

The trial was conducted in the R&D farm, Gummidipoondi, with 35 day old broiler birds (breed-Vencobb 400). The details of treatment groups are given in Table 10.

TABLE 10
Treatment groups used in trial

| | | | Dosage |
|---|---|---|---|
| Treatment Group | Diet | Enzyme | (g/ton of feed) |
| Control 1 | *Normal Diet | — | — |
| Control 2 | **Reformulated Diet | — | — |
| | (negative control) | | |
| Treatment 1 | **Reformulated Diet | Prototype 3 | 500 |
| Treatment 2 | **Reformulated Diet | Prototype 4 | 250 |

*Normal diet: Diet was formulated based on the nutrient requirements of Vencobb 400 broilers⁷
**Reformulated diet: AME value was reduced by 80 kcal/kg feed by reducing the quantity of rice bran oil and increasing de-oiled rice bran (DORB) in the feed formulation

Feed composition of the broiler diet used in the trial is shown in Table 11 and the nutrient composition is shown in Table 12.

TABLE 11
Feed composition of broiler diet

Composition
(kg/100 kg)

| | Normal | Reformulated |
|---|---|---|
| Ingredients | Diet | Diet |
| Maize | 58.91 | 58.469 |
| Soybean meal 45% | 29.906 | 29.363 |
| Rice Bran Oil | 4.569 | 3.539 |
| DORB | 3.000 | 5.000 |
| Calcite powder | 1.543 | 1.536 |
| Dicalcium phosphate | 0.898 | 0.886 |
| DL-Methionine | 0.267 | 0.262 |
| L-Lysine | 0.257 | 0.260 |
| Salt | 0.250 | 0.250 |
| Sodium Bicarbonate | 0.150 | 0.150 |
| Trace minerals | 0.100 | 0.100 |
| Choline chloride | 0.100 | 0.100 |
| L-Threonine | 0.050 | 0.050 |
| Vitamin Premix | 0.025 | 0.025 |
| Kemzyme PG | 0.010 | 0.010 |
| Total in kg | 100 | 100 |

TABLE 12
Nutrient Composition (theoretical) of broiler diet

Nutrient Value

| Nutrients | Normal | Reformulated |
|---|---|---|
| Metabolizable Energy (kcal/kg) | 3100 | 3020 |
| Crude Protein % | 19 | 19 |
| Crude Fiber % | 3.92 | 4.16 |
| Ether Extract % | 7.04 | 6.03 |
| Calcium % | 0.88 | 0.88 |
| Available Phosphorus % | 0.42 | 0.42 |

In the current trial, 48 birds were divided into 4 groups, with 12 birds per group. The birds were initially weighed and housed individually in wire cages with feeders and drinkers. Excreta collection trays were placed below each cage to collect the excreta. For the first 5 days of the trial, the birds were fed ad libitum feed (normal mash feed) for adaptation. This was followed by a 24 hour period of starvation in order to empty the gut contents. Each bird was then fed with 50 g of the respective test feed. Ad libitum water was provided throughout the trial period. Excreta was collected separately for individual birds for the next 36 hours, then dried for 48 hours at 70° C. in an oven, weighed and ground to pass through 1 mm mesh. The gross energy (GE) of the collected excreta and test feed samples were measured using adiabatic bomb calorimeter (Parr, United States of America). AME was determined using GE of excreta and feed as given in Equation 1.

Statistical analysis. Mean values were calculated for each treatment group. One way Analysis of Variance (ANOVA) was performed using STATGRAPHICS Plus 5.1 software to study the significance between different groups.

### Results

In the current trial, the AME value of the positive control group was 3002 kcal/kg. The negative control group was observed to have an AME of 2917 kcal/kg, which is 85 kcal/kg less than that of positive control group. This reduction in metabolizable energy observed in the negative control diet is found to be in line with the theoretical value (80 kcal/kg). These data confirms the effectiveness of feed formulation.

Prototype 3 administered at 500 g/ton of feed improved AME by 72 kcal per kg feed in broiler birds, whereas Prototype 4 supplemented at 250 g/ton dosage improved AME by 69 kcal/kg feed. Both the treatment groups showed an equivalent improvement in AME which was statistically significant compared to the negative control group (P<0.05). Improvements in AME observed with prototypes 3 and 4 matched the AME of positive control group. All these results are shown in FIG. 4.

In the current study, it was observed that the feed treated with Prototype 4 at the dosage of 250 g/ton has 11,017 units of xylanase per kg of feed; whereas, Prototype 3 administered at 500 g/ton has 19,966 units of xylanase per kg of feed (Table 9b). It is also to be noted that the concentration of other main chain degrading enzymes like cellulase, glucanase and amylase in Prototype 4 treated feed are twice less than that of Prototype 3. However, both the treatment groups have approximately 40 U of FAE treated per kg of feed. Even though the concentration of main chain degrading enzymes are less in feed treated with Prototype 4, presence of equal units of FAE has shown similar AME improvement in both the groups (FIG. 4). FAE by being synergistic with xylanase, is believed to aid the main chain hydrolases in degrading the plant cell wall by breaking the ferulate linkages and this is reported to improve AME of broiler birds. From the data, it is very evident that the dosage of main chain degrading enzymes can be reduced with the usage of FAE.

### Example 3

### FAE Improves Digestibility of High Fiber Diets

### Materials and Methods

Metabolic Trials.

AME trials were conducted in the R&D farm, Gummidipoondi, in two sets to check the efficacy of the above mentioned prototypes.

AME Trial.

This was conducted in 182 day old layer cockerels (breed—BV 300). In this trial, Prototypes 3 and 4 were evaluated. In these prototypes, FAE was included in the enzyme formulation to enhance the fiber digestion and also to work in synergy with xylanolytic and cellulolytic enzymes. To check the efficacy of Prototypes 3 and 4, a higher quantity of substrate (NSP) was required in the diet, so layer birds were used for the AME study. In this study, the crude fiber level of layer diet was raised up to 6.2% to provide enough substrate for the enzymes to release more energy (Tables 14 and 15). The details of treatment groups are given in Table 13.

TABLE 13
Treatment groups used in trial

| | | | Dosage |
|---|---|---|---|
| Treatment Group | Diet | Enzyme | (g/ton of feed) |
| Control 1 | *Normal Diet | — | — |
| Control 2 | **Reformulated Diet | — | — |
| | (negative control) | | |
| Treatment 1 | **Reformulated Diet | Prototype 3 | 500 |
| Treatment 2 | **Reformulated Diet | Prototype 4 | 250 |

*Normal diet: Diet was formulated based on the nutrient requirements of BV 300 layers14
**Reformulated diet: AME value was reduced by 80 kcal/kg feed by reducing the quantity of soya and maize, and increasing DORB, sunflower meal and broken rice in the feed formulation

TABLE 14
Feed composition of layer diet used in trial

| | Composition (kg/ton) | |
|---|---|---|
| Ingredient | Normal Diet | Reformulated Diet |
| Maize | 386 | 300 |
| Soya Meal | 117 | 83 |
| Broken Rice | 200 | 254 |
| De-oiled Rice Bran (DORB) | 80 | 100 |
| Sunflower Meal | 40 | 80 |
| Rapeseed Meal | 30 | 30 |
| Calcite | 20 | 20 |
| Stone Grit | 50 | 50 |
| Meat and Bone meal (MBM) | 30 | 30 |
| Dicalcium phosphate (DCP) | 2 | 2 |
| Fish Meal | 40 | 50 |
| Salt (sodium chloride) | 2 | 2 |
| DL-Methionine | 1 | 1 |
| L-Lysine | 0.5 | 0.5 |
| Soda Bicarbonate | 1 | 1 |
| Trace Minerals | 1 | 1 |
| Toxfin | 1 | 1 |
| Choline Chloride | 0.5 | 0.5 |
| Liver Tonic | 0.5 | 0.5 |
| Vitamin Premix | 0.25 | 0.25 |

TABLE 15
Nutrient composition (calculated) of layer diet used in trial

Nutrient value

| Nutrients | Normal Diet | Reformulated Diet |
|---|---|---|
| ME Kcal/Kg | 2590 | 2510 |
| Crude Protein % | 16.1 | 16.1 |
| Crude Fiber % | 5.06 | 6.16 |
| Ether Extract % | 2.20 | 2.03 |
| Calcium % | 3.07 | 3.00 |
| Available Phosphorous % | 0.52 | 0.51 |

In layer cockerel trials, 48 birds were divided into 4 groups, with 12 birds per group. The birds were initially weighed and housed individually in wire cages with feeders and drinkers. Excreta collection trays were placed below each cage to collect the excreta. For the first 5 days of the trial, the birds were fed ad libitum feed (normal mash feed) for adaptation. This was followed by a 24 hour period of starvation in order to empty the gut contents. Each bird was then fed with 50 g of the respective test feed. Ad libitum water was provided throughout the trial period. Excreta was collected separately for individual birds for the next 36 hours, then dried for 48 hours at 70° C. in an oven, weighed and ground to pass through 1 mm mesh. The gross energy (GE) of the collected excreta and test feed samples were measured using Parr adiabatic bomb calorimeter. AME was determined using GE of excreta and feed as given in Equation 1.

Statistical Analysis.

Mean values were calculated for each treatment group. One way Analysis of Variance (ANOVA) was performed using STATGRAPHICS Plus 5.1software to study the significance between different groups.

### Results

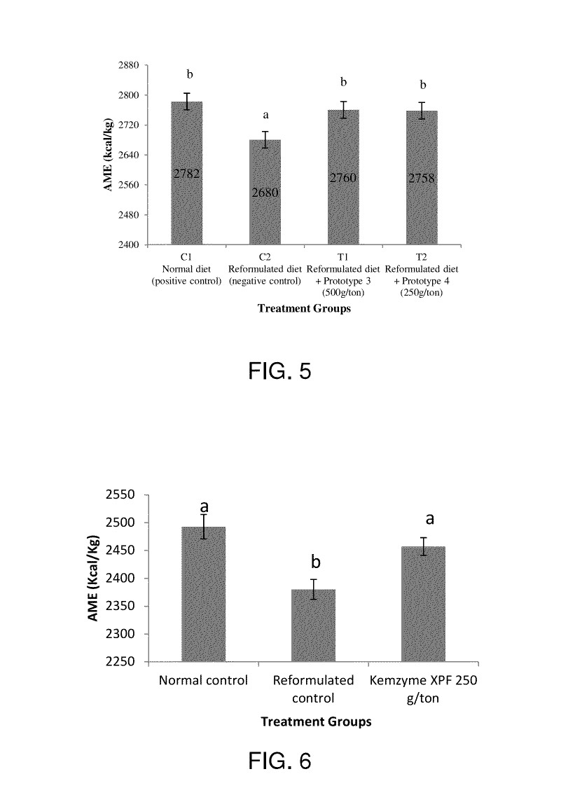

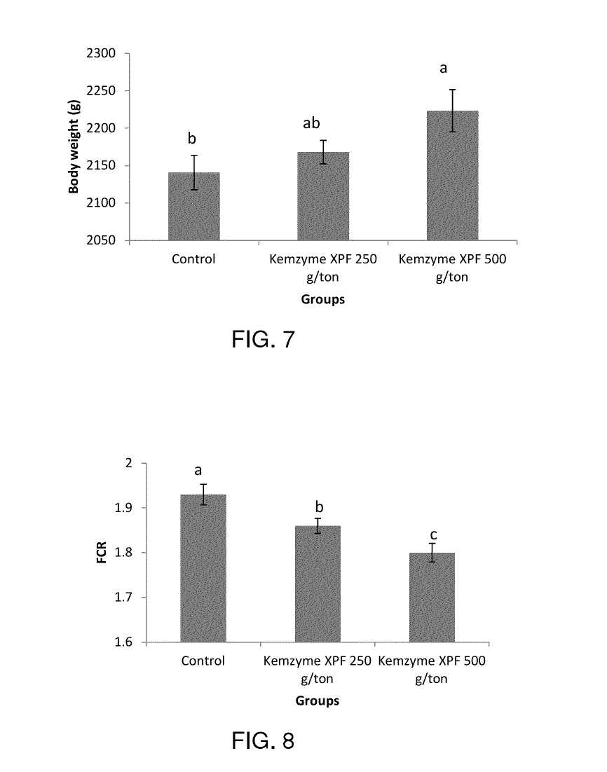

Prototype 3 administered at 500 g/ton of feed improved AME by 80 kcal per kg feed in layer cockerel birds, whereas, Prototype 4 supplemented at 250 g/ton dosage improved AME by 78 kcal/kg feed. Both the treatment groups showed an equivalent improvement in AME which was statistically significant compared to the negative control group (p<0.05). Improvements in AME observed with prototypes 3 and 4 matched the AME of positive control group. All these results are shown in FIG. 5.

### Example 4

### FAE Improves Apparent Metabolizable Energy of Laying Hens Materials and Methods

Kemzyme® XPF is a blend of amylase, NSPases and ferulic acid esterase (FAE) (Kemin Industries, Inc., Des Moines, Iowa).

Metabolic Trial.

The trial was conducted in the R&D farm, Gummidipoondi, with 182 day old layer cockerels (breed—BV 300). The details of treatment groups are given in Table 16.

TABLE 16
Treatment groups used in trial

| Treatment | | | Dosage |
|---|---|---|---|
| Group | Diet | Enzyme | (g/ton of feed) |
| Control 1 | *Normal Diet | — | — |
| Control 2 | **Reformulated Diet | — | — |
| | (negative control) | | |
| Treatment | **Reformulated Diet | Kemzyme ® XPF | 250 |
| 1 | | | |

*Normal diet: Diet was formulated based on the nutrient requirements of BV 300⁷
**Reformulated diet: AME value was reduced by 74 kcal/kg feed and crude fiber was increased by 1% by reducing the quantity of maize, meat & bone meal (MBM) & soya meal and increasing de oiled rice bran (DORB), Sunflower meal & Rapeseed meal in the feed formulation

Feed composition of the layer diet used in the trial is shown in Table 17 and the nutrient composition is shown in Table 18.

TABLE 17
Feed composition of layer diet

Composition (g/kg)

| Ingredients | Normal feed | Reformulated feed |
|---|---|---|
| Maize | 300 | 250 |
| Soya Meal 45% | 97 | 86 |
| Broken Rice | 264 | 278 |
| DORB | 110 | 136 |
| Sunflower Meal | 80 | 100 |
| Rapeseed Meal | 30 | 40 |
| Calcite/LSP | 17.75 | 20.6 |
| Stone Grit | 30 | 30 |
| MBM | 20 | 8 |
| Dicalcium phosphate | 4 | 4 |
| Fish Meal 40% | 40 | 40 |
| Salt | 2 | 2 |
| DL-Methionine | 0.5 | 0.44 |
| L-Lysine | 0.5 | 0.75 |
| Soda Bicarbonate | 1 | 1 |
| Trace Minerals | 1 | 1 |
| Toxfin | 1 | 1 |
| Choline Chloride | 0.5 | 0.5 |
| Liver Tonic | 0.5 | 0.5 |
| Vitamin Premix | 0.25 | 0.25 |
| Total | 1000 | 1000 |

TABLE 18
Nutrient Composition (theoretical) of layer diet

| | Nutrient Value | |
|---|---|---|
| Nutrient | Normal Diet | Reformulated Diet |
| Metabolizable Energy (kcal/kg) | 2598 | 2524 |
| Crude Protein % | 16.5 | 16.5 |
| Crude Fiber % | 6.4 | 7.3 |
| Fat % | 1.95 | 1.76 |
| Calcium % | 2.3 | 2.3 |
| Available Phosphorous % | 0.35 | 0.35 |

In the current trial, 48 birds were divided into 4 groups, with 12 birds per group. The birds were initially weighed and housed individually in wire cages with feeders and drinkers. Excreta collection trays were placed below each cage to collect the excreta. For the first 5 days of the trial, the birds were fed ad libitum feed (normal mash feed) for adaptation. This was followed by a 24 hour period of starvation in order to empty the gut contents. Each bird was then fed with 50 g of the respective test feed. Ad libitum water was provided throughout the trial period. Excreta was collected separately for individual birds for the next 36 hours, then dried for 48 hours at 70° C. in an oven, weighed and ground to pass through 1 mm mesh. The gross energy (GE) of the collected excreta and test feed samples were measured using adiabatic bomb calorimeter (Parr, United States of America). AME was determined using GE of excreta and feed as given in Equation 1.

Statistical Analysis.

Mean values were calculated for each treatment group. One way Analysis of Variance (ANOVA) was performed using STATGRAPHICS Plus 5.1 software to study the significance between different groups.

### Results

In the current trial, AME value of positive control group was 2493 kcal/kg; whereas, the negative control group was observed to have an AME of 2380 kcal/kg, which is 113 kcal/kg less than that of positive control group. The reduction in metabolizable energy in the negative control diet was observed to be higher than the theoretical value (74 kcal/kg). This can be attributed to the variation in the actual nutrient values against the theoretical values of the raw materials used.

Kemzyme® XPF administered at 250 g/ton of feed improved AME significantly (P<0.01) over negative control and was comparable to that of positive control (FIG. 6). Kemzyme® XPF improved AME by 77 kcal/kg feed in layer cockerel birds. The presence of ferulic acid (FA) cross links in the cell wall, limits the biodegradability of cereal based diets. FAE has the ability to hydrolyze the ester bond between the xylan polysaccharide and the ferulate or diferulates present in the plant cell walls. FAE by being synergistic with xylanase, is believed to aid the main chain hydrolases in degrading the plant cell wall by breaking the ferulate linkages.

### Example 5

### FAE Improves Performance of Broiler Birds

### Materials and Methods

Trial Design.

The trial was conducted at Tropical Institute of Livestock Management and Animal Husbandry (TILMAH), Gurgaon. The trial included one control and two different treatment groups. The trial design and dosage levels of the test products are shown in Table 19.

TABLE 19
Details of experimental groups and dosage of test samples

| Groups | Product | Supplier | Dosage (g/ton) |
|---|---|---|---|
| Control | — | — | — |
| ^{$}Treatment 1 | Kemzyme ® XPF | Kemin Agrifoods | 250 |
| | | India | |
| ^{$}Treatment 2 | Kemzyme ® XPF | Kemin Agrifoods | 500 |
| | | India | |

^{$}All the treatment groups had reformulated diet with 70 kcal/kg metabolizable energy (ME) reduction

The farm trial was conducted with commercial hybrid broiler chicks (VenCobb 400 strain) for a period of six weeks. A day old chicks were procured from Choice poultry breeding farm, Jind, Haryana, India. All the birds were obtained from the same breeder flock for the trial. Then they were divided into 3 groups with 180 birds in each. Each group was further divided into 9 replicates and each replicate had 20 birds. The experiments were conducted in floor pens of 5 ft×5 ft (1.25 sq.ft/bird). Completely randomised design was adopted to minimize the effect of environment and management on different groups. All the broilers were reared under similar management conditions over a deep litter (saw dust bedding) system throughout the experimental period. Saw dust was processed by spreading the material in the sun for about 4 days, after which bleaching powder, lime powder and Omnicide (disinfectant) were sprinkled and used for bedding.

Experimental Diet.

In the present trial, the growth period of broilers has been divided into three phases: pre starter (0-7 days), starter (8-21 days) and finisher (22-42 days) phases which is normally practiced in Indian commercial broiler farms. The feed was manufactured at Ami Chand Makhan Lal Feeds Pvt Ltd., Gurgaon. The feed was formulated based on the nutrient requirements of VenCobb 400 broilers. All the ingredients were milled using a hammer mill and passed through 3 mm sieve for pre-starter, starter and finisher diets. All the ingredients as per the formula were blended well to uniformity in a 1 tonne mixer. The feed was reformulated by reducing rice bran oil, maize & soya DOC (De-Oiled Cake) and by increasing rice polish in the formulation at different ratios during different phase of the trial. The treatment feeds were prepared by blending the respective test chemicals in the reformulated feed, labelled and transported to the farm for the trial. The details of the feed and nutrient composition of the experimental diets are shown in Table 20 and 21.

TABLE 20
Feed composition of diets used in trial

| | Composition (g/kg) | | | | | |
|---|---|---|---|---|---|---|
| | Normal Diet | | | Reformulated Diet | | |
| Ingredients | Prestarter | Starter | Finisher | Prestarter | Starter | Finisher |
| Maize 10% M/W | 534 | 544 | 623 | 528 | 559 | 598 |
| Soya DOC 45% | 321 | 263 | 200 | 317 | 260 | 194 |
| Rice Polish 12% | 30 | 50 | — | 50 | 50 | 42 |
| Lime Stone Powder | 11.847 | 10.132 | 3.777 | 11.198 | 9.632 | 3.251 |
| Dicalcium Phosphate | 4.985 | 0.113 | — | 5.112 | 0.068 | — |
| DL-Methionine | 2.798 | 2.52 | 2.232 | 2.793 | 2.512 | 2.229 |
| L-lysine | 3.43 | 2.504 | 2.526 | 3.464 | 2.563 | 2.563 |
| Rapeseed DOC | 20 | 20 | 30 | 20 | 20 | 30 |
| Rice Bran Oil | 22 | 39 | 50 | 13 | 27 | 39 |
| MBM Standard 45% | 20 | 30 | 40 | 19 | 30 | 40 |
| Fish meal 44% | 20 | 30 | 40 | 20 | 30 | 40 |
| L-threonine | 0.565 | 0.312 | 0.504 | 0.566 | 0.318 | 0.507 |
| Salt | 2.025 | 1.769 | 1.511 | 2.017 | 1.757 | 1.5 |
| Sodium Bicarbonate | 1.75 | 1.75 | 1.75 | 1.75 | 1.75 | 1.75 |
| Choline chloride 60% | 1.5 | 1.3 | 1.1 | 1.5 | 1.3 | 1.1 |
| Ultra TM | 0.5 | 0.5 | 0.5 | 0.5 | 0.5 | 0.5 |
| Vitamin Premix | 0.5 | 0.5 | 0.5 | 0.5 | 0.5 | 0.5 |
| Bacitracin Methylene | 0.5 | 0.5 | 0.5 | 0.5 | 0.5 | 0.5 |
| Disalicylate (BMD) | | | | | | |
| Liver tonic | 0.5 | 0.5 | 0.5 | 0.5 | 0.5 | 0.5 |
| Salinomycin 12% | | 0.5 | 0.5 | | 0.5 | 0.5 |
| CMP | 1 | — | — | 1 | — | — |
| Kemzyme PG 5000 | 0.1 | 0.1 | 0.1 | 0.1 | 0.1 | 0.1 |
| (Phytase) | | | | | | |
| Toxin Binder | 1 | 1 | 1 | 1 | 1 | 1 |
| Total | 1000.00 | 1000.00 | 1000.00 | 1000.00 | 1000.00 | 1000.00 |

*Kemzyme XPF was added to the feed formulation at 250 g/ton or 500 g/ton depending on the respective treatment groups

TABLE 21
Nutrient composition (theoretical) of diets used in trial

| | Nutrient Values | | | | | |
|---|---|---|---|---|---|---|
| | Normal Diet | | | Reformulated diet | | |
| Nutrient | Prestarter | Starter | Finisher | Prestarter | Starter | Finisher |
| Crude Protein % | 22.5 | 21 | 19.5 | 22.5 | 21 | 19.5 |
| Metabolizable Energy | 3000 | 3125 | 3250 | 2930 | 3055 | 3180 |
| (kcal/kg) | | | | | | |
| Calcium % | 0.94 | 0.92 | 0.88 | 0.94 | 0.92 | 0.88 |
| Available | 0.45 | 0.42 | 0.4 | 0.45 | 0.42 | 0.4 |
| Phosphorous % | | | | | | |
| Ether Extract % | 5.28 | 7.21 | 7.98 | 4.5 | 6.1 | 7.55 |

Farm Management.

All the experimental birds were provided with respective feed and water ad libitum throughout the experimental period. Lighting was provided for 24 hours a day throughout the trial period, which involves 12 hrs of natural lighting and 12 hrs of lighting using incandescent lamps. The experiment was conducted in the summer (April to May) and the temperature of the shed ranged between 38-40° C. and relative humidity ranged between 70-75% throughout the trial period.

All the birds were provided medications against diseases common to the area as per the standard practice: Tylosin—2nd, 3rd, 4th, 21st and 22nd days (in drinking water); New Castle Vaccine (F strain)+IV—5th day (intra ocular route); Georgia vaccine, Gumboro Disease-14th day (intra ocular route); and Lasota vaccine—23rd day (in drinking water).

Parameters Measured.

The parameters measured during the trial were weekly live weight, weekly gain in weight, weekly feed consumption, and feed conversion ratio (FCR).

Performance Parameters.

Birds from each treatment were weighed individually on a weekly basis. The average weight of the birds was calculated replicate wise and finally the average weight of the birds per treatment was calculated. The feed consumption was measured per replicate on weekly basis and finally the average feed consumption per treatment was calculated. Feed conversion ratio was calculated with the average bird weight gain and the feed consumption of the respective group (feed consumption/weight gain).

Statistical Analysis.

Mean values were calculated for each treatment group. One way Analysis of Variance (ANOVA) was performed using STATGRAPHICS Plus 5.1 software to study the significance between different groups. The data were analyzed by Least Significant Difference (LSD) method and differences at P<0.05 are considered significant.

### Results

Cereal grains are the important source of energy in poultry diets and are rich in NSPs. These are the complex and heterogeneous groups of macromolecules, containing diverse polysaccharides as building blocks. Poultry do not have enzymes to break down these NSP, which limits the digestibility of animal feed. Hence exogenous supplementation of NSP degrading enzymes is necessary to improve the digestibility of NSPs.

In the present study, the normal broiler diet was formulated with maize, rice bran oil, rapeseed DOC and rice polish as the sources of ME, and soybean meal as main protein source. In the reformulated diet, AME value was reduced by 70 kcal/kg feed by reducing the quantity of rice bran oil & maize and by increasing rice polish in the feed formulation. In our previous study, we have reported that Kemzyme® XPF has improved AME of poultry diets by 70-80 Kcal/kg. The product was found to improve AME of layer diets significantly (P<0.05) over negative control group.

In the present study, the addition of Kemzyme XPF at 250 g/ton and 500 g/ton showed a dose response on body weight and FCR of birds at 42 days of age. It is to be noted that, the groups treated with Kemzyme XPF had 70 Kcal/kg ME reduced compared with control group. The addition of Kemzyme XPF to the reformulated diet with reduced energy showed higher body weight compared to control. The addition of Kemzyme XPF at 500 g/t had significantly higher body weight compared to control group. The addition of Kemzyme XPF reduced the FCR of the birds, compared to control at 42 days of age. The groups supplemented with Kemzyme XPF at 250 g/ton and 500 g/ton lowered FCR by 7 points and 13 points respectively, compared to the control group. The results of the current study evidences that, addition of FAE along with main chain degrading enzymes improves performance of broiler birds.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

We claim:

1. A method of improving the apparent metabolizable energy from a diet and performance in an animal, comprising the step of adding an efficacious amount of a ferulic acid esterase to the diet supplemented with or without main chain degrading enzymes.

2. The method of claim 1, wherein the main chain degrading enzymes are selected from the group consisting of cellulase, xylanase, glucanase and amylase.

3. The method of claim 1, wherein the efficacious amount of ferulic acid esterase ranges from 20 U/kg to 200 U/kg of feed.

4. The method of claim 1, wherein the animal is a monogastric.

5. The method of claim 4, wherein the monogastric is selected from the group consisting of poultry and swine.

6. A method of reducing the main chain degrading enzymes necessary to extract a given amount of the apparent metabolizable energy from a diet in an animal, comprising the step of adding an efficacious amount of a ferulic acid esterase to the diet.

7. The method of claim 6, wherein said reduction is by between 20% and 80%.
